(19) United States
(12) Patent Application Publication
(10) Pub. No.: US 2005/0118701 A1
(43) Pub. Date: Jun. 2, 2005

(54) Large scale methods of producing adenovirus and adenovirus seed stocks

(76) Inventors: Weichang Zhou, Livermore, CA (US); Liangzhi Xie, North Wales, PA (US); Nedim Emil Altaras, Harleysville, PA (US); John G. Aunins, Doylestown, PA (US)

Correspondence Address: MERCK AND CO., INC, P O BOX 2000, RAHWAY, NJ, 07065-0907, US

(21) Appl. No.: 10/509,302

(22) PCT Filed: Mar. 27, 2003
(86) PCT No.: PCT/US03/09274

Related U.S. Application Data
(60) Provisional application No. 60/368,706, filed on Mar. 29, 2002.

(51) Int. Cl. C12N 7/02

(52) U.S. Cl. 435/239

(57) ABSTRACT

A process for large scale virus production is disclosed, especially large scale adenovirus production. The methods described are preferably adapted to suspension culture of mammalian host cells in a large scale bioreactor where gas sparging becomes essential to provide adequate aeration through the duration of the culture. This methodology includes an elevated concentration of a compound which protects host cells from the shearing effects of gas sparging and agitation as well as utilizing virus seed stocks which have been generated free of cell lysis reagents. The invention also relates to methods of producing these virus seed stocks, which are scaleable and the resultant unclarified virus seed stocks which are concentrated to reduce storage volume for infection of large scale culture and which are free of cell lysis components, such as the detergent Triton X-100 or Polysorbate 80.

### CROSS-REFERENCE TO RELATED APPLICATIONS

This application claims benefit, under 35 U.S.C. §119(e), to U.S. provisional application 60/368,706 filed Mar. 29, 2002.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to a process of utilizing sparged cultures for large scale virus production which relies on the use of concentrated virus seed stocks which are free of cell-lysing components as well as the use of increased amounts of shear-protection reagents to reduce host cell damage, especially after infection of the culture with virus. The present invention also relates to clarified or unclarified virus seed stocks which are prepared and formulated to be essentially free of cell-lysis components.

### BACKGROUND OF THE INVENTION

Advances in the areas of the use of recombinant viral vectors for gene therapy and DNA vaccination applications have created a need for large scale manufacture and purification of clinical-grade virus. One such family of viruses are the adenoviruses. The adenoviruses are grouped within the family Adenoviridae, which are split into the genus Aviadenovirus (birds) and Mastadenovirus (human, simian, bovine, equine, porcine, ovine, canine and opossum). A review of the family Adenoviridae can be found in Fundamental Biology, 3^{rd} Ed., Fields, B. N., Knipe, D. M., and Howley, P. M., Ed., at Chapter 30, pp. 979-1016 (1996), which is hereby incorporated by reference. Of specific interest in gene vaccination and/or gene therapy applications is the use of a 1^{st} or 2^{nd} generation replication incompetent adenovirus, crippled by E1 or further deletions, including “gutless” adenovirus vectors. The adenovirus genome is generally associated with benign pathologies in humans, and the genomic organization of the virus has been well studied since its discovery in the early 1950s. In addition, the genome is amenable to manipulation, depending on the strategy utilized to construct the respective vector. A replication-incompetent virus (such as an E1/E3 deleted Ad5gag vector expressing a HIV gag transgene, as exemplified herein) requires a cell line which complements the deletions. Any such cell line may be used to generate recombinant virus vectors, with preferred, but not limiting, cell lines including 293 cells and PER.C6™ cells. To this end, numerous 1^{st} generation recombinant adenovirus vectors have been described in the literature (e.g., see Bett, et al., 1994, Proc. Natl. Acad. Sci. 91:8802-8806; WO 01/02607 and WO 02/22080). “Gutless” adenoviral vectors are a 2^{nd} generation adenoviral vector generally devoid of viral protein-coding sequences, frequently with viral proteins supplemented in trans by a helper virus (often an E1-deleted adenovirus) grown with the helper-dependent (HD) adenovector in a packaging cell line (e.g., PER.C6™). Absent viral proteins, these viral vectors can, in the alternative, be supplemented in trans by a cell line capable of expressing the structural and functional adenoviral proteins necessary for successful replication, packaging and rescue. In view of the increased popularity of these viral vectors and the ultimate need to prepare commercial scale quantities of either a viral vector based vaccine or gene therapy vehicle, it has become essential to develop more efficient qualitative and quantitative methodology for production of commercial grade recombinant adenovirus vectors.

Adenovirus production, especially in serum-free PER.C6™ cell culture, has not been practiced routinely at large-scale, where gas sparging is required for oxygenation. At small-scale, surface aeration instead of gas sparging is generally adequate to provide sufficient oxygen supply and CO₂ removal and hence the sensitivity to sparging can be circumvented. At large-scale, gas sparging is often necessary for oxygenation and CO₂ removal and hence resolving issues with sensitivity to gas sparging for virus infected cell cultures are necessary to allow the use of this convenient oxygen method for large-scale bioreactor design and virus production. To protect cells from damage by gas sparging, a shear-protecting reagent such as Pluronic F-68 may be used.

One aspect of preparing for commercial scale production of adenovirus is the production of virus seed stocks. The production of virus seed stocks in cell cultures in and of itself are known in the art (see, e.g., U.S. Pat. Nos. 4,055,466, 4,072,565, 4,080,258, and especially U.S. Pat. No. 5,994,134 as an example relating to adenovirus. The '134 patent discloses the use of a microcarrier reliant culture system to produce large amounts of virus, including adenoviruses.

Viral seed stocks are prepared by recovery of extra- and/or intracellular viruses of infected cell culture under aseptic conditions. Intracellular viruses can be recovered through cell lysis, and virus concentration can be achieved either by ultrafiltration or separation of virus-containing cells prior to lysis. In general, buffers containing detergents such as Polysorbate 80 or Triton X-100 or freeze/thaw are used to achieve cell lysis for virus release. The cell lysis procedure using detergents allows significant cell lysis and virus release. It is simple, scalable and widely used, but the resulting virus seeds will contain a detergent(s) that can cause cell membrane damage even at a diluted concentration. This might detrimentally affect their subsequent use for virus propagation, depending on culture conditions. On the other hand, the cell lysis procedure using freeze/thaw, even though it is simple and mostly used at small-scale, can not be easily scaled up. Thus, it is not practical for preparation of large-scale virus seeds, which are used to infect cultures at large-scale, such as commercial scale preparations which will run from 1,000 to 50,000 L, or even larger.

U.S. Pat. No. 6,186,941 discloses a method of generating adenovirus stocks which relies on a continuos perfusion of fresh medium into the culture prior to infection.

U.S. Pat. Nos. 6,146,891 and 6,168,944 disclose methods of cell culture and virus production which rely on adhesion of cells to mircocarriers during the culture process.

U.S. Pat. No. 5,837,520 (see also Huyghe et al., 1995, Human Gene Therapy 6: 1403-1416) discloses a method of purifying adenovirus which comprises treating the cell lysate with a nuclease, followed by (1) anion exchange and (2) metal ion chromatography.

U.S. Pat. No. 6,261,823 B1 disclosed a method of purifying adenovirus which comprises subjecting the adenovirus preparation to anion exchange chromatography followed by size exclusion chromatography.

U.S. Pat. No. 6,194,191 discloses methods of purifying adenovirus using low perfusion rates during cell culture, a detergent lysis step, and/or a single chromatography step.

One of the most important factors in the design of mammalian cell bioreactor processes is oxygen supply to the cells. Various methods are used at the research scale, including surface aeration, membrane aeration, and gas sparging. Gas sparging is the method of choice for aeration in most large-scale cell culture bioreactor processes due to high oxygen transfer rates and simplicity of reactor design and scale-up. However, it is known that cell to bubble interactions damage shear-sensitive mammalian and insect cells, and this remains an important issue in the large scale production runs that are the central to the teachings of the present invention. It is thought in the art that a mechanism of sparge-induced cell damage is the shear associated with bubble rupture at the gas-liquid interface. Attachment to bubbles in the bulk liquid localizes cells in the liquid layer surrounding the bubble, resulting in cell death when the bubble bursts. Decreased bubble diameter and increased gas flow rates have been shown to increase cell damage in bioreactors. The majority of studies on mitigating the damaging effect of sparging on shear sensitive cell cultures have focused adding components to the culture medium. Various medium additives have been identified that may affect cell-to-bubble attachment, foaming, and physical properties of the bulk liquid. Common methods of improving the viability of sparged cultures include lowering the rate of cell attachment, decreasing surface tension, and increasing liquid viscosity. The effect of foam stability on viability in sparged reactors is not yet clear. Serum and/or surfactants are often used to improve cell growth in sparged environments. Increasing concentrations of serum in culture medium is known to decrease cell damage in sparged reactors both physically and biologically. It is hypothesized that this is the result of reduced cell attachment, decreased turbulence due to increasing medium viscosity, decreased plasma membrane fluidity, and reduced shear sensitivity of cells in culture. Serum has been shown to stabilize foam in sparged cultures. Unfortunately, increasing serum concentrations is not a viable option in the serum-free cell culture processes prevalent in the current regulatory environment. Surfactants such as polyvinyl alcohols have been shown to decrease cell-to-bubble attachment but have different effects on foam stability. The protective mechanism for these polymers is not yet clear. In addition to reducing cell attachment to bubbles, they may also lower the surface tension of the medium, providing protection under sparging conditions.

The block copolymer of Pluronic®F-68 is made up of a hydrophobic center (polyoxypropylene block) surrounded by two hydrophilic ends (polyoxyethylene blocks). Pluronic®F-68 has been widely used as a protectant in insect and mammalian cell cultures. Pluronic®F-68 has been shown to decrease cell-to-bubble attachment and surface tension in sparged cultures. Other hypotheses for its protective effect are direct interaction of PF68 with the cell membrane and the formation of a stable foam layer, which allows cells to drain from the film near bursting bubbles. Most studies of PF68 have been conducted at a concentration of 0.1% in the medium, though its effect has been demonstrated to be concentration dependent. No toxicity has been observed at concentrations up to 0.5% in sparged, baculovirus-infected Spodoptera frugiperda Sf9 cultures. Murhammer and Goochee (1988, Bio/Technology 6:1411-1418) tested the use of several Pluronic® polymers of varying structure and found that those with the highest hydrophilic-lipophilic balance (HLB) to be the most protective (those with the lowest HLB actually lysed cells).

Little is known about the shear-sensitivity of infected mammalian cell cultures. Virus-infected microcarriers cultures have been shown to be vulnerable to agitation damage, but all other evidence on the effect of virus infections is indirect. To date there is no information available in the literature on PER.C6™ cells uninfected or infected with adenovirus. Late stage adenovirus infections can cause cell lysis, but it is unknown whether or not virus infections would exacerbate cell damage from sparging. Therefore, despite these reports, there remains a need for the development of a large-scale process for producing adenoviral seed stocks which will compliment the stringent quantitative and qualitative requirements of commercial scale adenovirus production for vaccine and/or gene therapy applications. The present invention addresses and meets these needs by disclosing a scaleable method of generating a high quality, concentrated, clarified or unclarified adenovirus seed stock free of any cell lysing agents which can then be utilized in an improved large scale cell culture process which is free of the deleterious effects caused by the presence of such cell lysis agents in large-scale adenovirus production.

### SUMMARY OF THE INVENTION

The present invention relates to a process for large scale virus production. The methods disclosed herein are preferably adapted to suspension culture of mammalian host cells in a large scale bioreator where gas sparging becomes essential to provide adequate aeration through the duration of the culture. The large scale methods of the present invention preferably include the use of virus seed stocks for infecting host cell culture which have been generated free of cell lysis reagents, such as the detergent Triton X-100 or Polysorbate 80 (C₁₈H₃₃O₂). It is disclosed herein that infecting the cell culture with such a virus seed stock while also including increased levels of a respective shear-protecting compound or a combination of more than one shear-protecting compound in conjunction with sparging results in a reproducible, commercially viable large scale virus production process. A preferred but by no means limiting shear-resistant compound is a surfactant such as Pluronic®F-68, a non-ionic detergent block copolymer which has been shown to be an effective shear resistant compound in serum free mammalian growth culture media. It is shown herein a shear-resistant/protective compound may be added at any time during the cell culture process up to the time of infection with the respective virus seed. Therefore, while it is a decision of convenience to add a shear-protective compound(s) pre-infection while cells continue to grow in the culture medium (e.g., from the start of the large scale culture), it is certainly reasonable and well within the scope of the disclosed methodology to add all or the bulk of the shear protective agent at the time of or at a time reasonably near the time of seeding the culture with the respective virus seed stock.

A specific embodiment of the present invention relates to a process for the large scale production of adenovirus within a sparged culture system. As noted above, the methods disclosed herein as applied to any wild type, modified or recombinant adenovirus serotype are preferably adapted to suspension culture of mammalian host cells in a large scale bioreactor with gas sparging to provide adequate aeration. This large scale method also preferably includes the use of adenovirus seed stocks which have been generated free of cell lysis reagents and the inclusion of a shear-protecting compound to offset deleterious effects by gas sparging conditions on host cells. Again, a preferred but by no means limiting shear-protecting compound is a surfactant, such as the surfactant Pluronic®F-68.

A particular embodiment of the present invention is exemplified herein, namely the large-scale production of a recombinant Adenovirus encoding HIV-1 transgene gag (MRKA5gag, as described in WO 02/22080, which is hereby incorporated by reference) in serum-free cell culture under gas sparging conditions. More specifically, a combination of steps results in an improved large scale scheme for producing adenovirus, namely (1) infecting the cell culture with virus seeds which are free of any cell lysis components, and (2) adding Pluronic®F-68 to the culture at a concentration of at least about 1 g/L or higher to improve process robustness. The inclusion of these two steps allow for the production of robust virus lots that may be reproduced for large scale production runs, including bioreactor scale-up for adenovirus production in lots of 10,000 L or higher.

The present invention also relates to methods of producing virus seed stocks, especially adenovirus seed stocks, which are free of cell-lysis components. This portion of the invention comprises first inoculating and culturing host cells in a cell growth medium free of cell lysis components, infecting the host cells with an adenovirus of any serotype, either of a wild type, modified (e.g., such as an attenuated virus) or more likely, a recombinant form. The resulting adenovirus-infected host cells are cultured for an appropriate time and then the adenovirus is harvested from both infected host cells (intracellular adenovirus) as well as the cell culture medium (extracellular adenovirus) without the aid of any added cell-lysis component and without use of freeze/thaw. The adenovirus may be further processed in any number of ways that are known in the art. For example, the cell lysis component-free adenovirus may be concentrated, resulting in an unclarified, concentrated adenovirus seed stock free of any cell-lysis reagents. Alternatively, clarification and virus release may be a combined step, followed by concentration of the remaining virus volume to generated a clarified virus seed stock free of any cell-lysing components. Other potential avenues for generating the final virus seed stocks of the present invention include but are in no way limited to (1) a lysis/concentration stage which results in an unclarified virus seed (2) a lysis/concentration stage followed by clarification via any known methodology, such as filtration or centrifugation, which results in a clarified virus seed; (3) lysis/clarification followed by concentration of the clarified virus seed, and/or (4) lysis/clarification for unconcentrated virus seed. The above mentioned protocols are conducted under aseptic conditions. Any non-aseptic process is followed by a sterile filtration step. In other words, the inventors prefer to engage in aseptic processing when generating a virus seed stock. However, it is certainely within the purview of the present teachings for the skilled artisan to practice non-aspectic steps during the virus recovery process and then follow up with a sterile filtration step, or in the alternative, to generate a non-aseptic seed in itself. And as noted above, any method or combination of methods may be used to prepare the virus seeds generated free from cell lysis components, including but not limited to a clarified virus seed, an unclarified virus seed, or even an unclarified virus seed which is then subject to a later clarification step.

A preferred methodology for generating virus seed stocks free of any cell-lysis components, including but not limited to adenovirus seed stocks, is the use of a hollow fiber ultrafiltration device to simultaneously harvest viruses from the virus infected cell culture, lyse the host cells mechanically, and concentrate the available virus particles, such as adenovirus particles, providing for a concentrated, unclarified virus seed stock useful in infecting large scale cell culture for virus production. The unclarified virus seed stock may be further clarified. As noted in the previous paragraph, by no means is this portion of the invention limited to method of producing final virus seed stocks which remain unclarified. In other words, this portion of the invention may include concentrated virus seed stocks which are unclarified, clarified, or unclarified stocks which are later subjected to clarification.

The present invention relates to virus seed stocks which are devoid of any cell-lysing components.

The present invention relates to virus seed stocks which are devoid of any cell-lysing components, including but not limited to adenovirus seed stocks.

The present invention also relates to virus seed stocks which are devoid of any cell-lysing components, including but not limited to adenovirus seed stocks, of which were generated by methods disclosed herein.

As noted above, a preferred embodiment of the present invention relates to adenovirus seed stocks which are devoid of any cell-lysing component. An adenovirus seed stock may be a stock of any wild type, modified (such as an attenuated form) and/or recombinant serotype of adenovirus known in the art.

It is an object of the present invention to provide for a method of large scale virus production which utilizes gas sparging but overcomes the deleterious effects of gas sparging by infecting the culture with virus seed stocks which have been generated free of cell lysis reagents and by adding to the culture increased levels of a shear-resistant compound, such as Pluronic®F-68, hydroxyethyl starch, derivatives of cellulose, serum, tryptosephosphate, polyvinyl alcohol (PVA), bovine serum albumin, polyethylene glycol (PEG), and/or dextran, or any combination of more than one to a reasonable combination thereof that provides protection against pre- and/or post-virus infection damage to host cells.

It is a further object to provide for clarified and/or unclarified virus seed stocks, such as adenovirus seed stocks, which are free of cell-lysis components, and associated methods to generate such clarified and/or unclarified virus seed stocks in large-scale.

As used herein, “L” is the abbreviation for—liters—.

As used herein, “VVM” is the abbreviation for—volumes of gas per volume of liquid per minute—.

As used herein, “TMP” is the abbreviation for—transmembrane pressure—.

As used herein, “PF-68” is the abbreviation for—Pluronic® F-68—.

As used herein, “LMH” is the abbreviation for—L/minute²/hour—.

As used herein, “vp” or “VP” means—viral particles—

As used herein, “vp/cell” means—viral particles per cell—

As used herein, a “shear-resistant compound” or “shear-protecting compound”, “shear-protecting reagent” and similar terms are used interchangeably and are meant to define compound or reagent which may be added to a cell culture medium to provide a level of protection against shearing forces which may be prevalent in large scale mammalian cell bioreactors, such as excessive agitation and gas sparging. A “shear-protecting compound”, includes but is by no means limited to compounds such as Pluronic®F-68, hydroxyethyl starch, derivatives of cellulose, serum, tryptosephosphate, polyvinyl alcohol (PVA), bovine serum albumin, polyethylene glycol (PEG), and/or dextran, as well as any combination thereof. These compounds offer protection from shear related cell damage for various mechanisms, but do not promote lysis of infected or non-infected host cells, such as detergents like Triton-As used herein, the term “large scale”, “commercial scale”, “pilot scale” or “manufacturing scale” and similar terms relate to culture of host cells, especially mammalian cells and most likely in bioreactors, which may have a total volume from about 10 L all the way up to 50,000 L, and beyond, with preferable large scale preparations coming from the 100 L to 20,000 L stage. These terms are meant, for the purpose of this disclosure, to distinguish and define a large scale mammalian cell culture as one in which gas sparging becomes a basic necessity in order to maximize virus production in these mammalian cell cultures. It is the requirement for gas sparging during pre- and post-infection that has led the inventors to create the improved culture conditions which lead to superior virus production.

As used herein, term “free of any cell-lysis components”, or any similar recitation, means that any composition mentioned throughout this specification, including but not limited to a cell culture growth medium or a clarified or unclarified virus seed stock, does not contain a concentration of a cell-lysis component which would have a deleterious effect on said composition. It will be understood to the skilled artisan that de minimus amounts of any such compound might be present in the composition, but because of the vast number of components within the composition, such as host cells or recovered virus particles, any deleterious effect that the cell-lysis compound would be negligible.

### BRIEF DESCRIPTION OF THE FIGURES

FIG. 1 shows flux and transmembrane pressure (TMP) profile during the single step cell lysis and concentration unit operation for preparation of a virus seed at lab-scale utilizing a hollow fiber ultrafiltration system.

FIG. 2 shows flux and transmembrane pressure (TMP) profile during the single step cell lysis and concentration unit operation for preparation of a virus seed at large-scale utilizing a hollow fiber ultrafiltration system.

FIG. 3 shows the flux and TMP profile throughout the clarification step for a lab scale run utilizing a 0.45 μm regenerated cellulose Sartorius membranes at lab scale.

FIG. 4 shows the Flux and TMP profile during the concentration unit operation for a lab scale preparation utilizing during concentration a 300 kDa PES Sartorius membrane at lab scale

FIG. 5 shows viable cell concentration (Exp. #3 of Example 4) in 2 L non-sparged bioreactors in regard to varying concentrations of Pluronic®F-68.

FIG. 6 shows viable cell concentration (Exp. #4 of Example 4) in 2 L sparged bioreactors with culture medium containing 1 g/L or 10 g/L Pluronic®F-68.

FIG. 7 shows the effect of virus buffer concentration on virus production in sparged reactors (Exp. #4 in Example 4).

FIG. 8 shows PER.C6™ cell growth in 2 L and 300 L bioreactors with and without sparging (error bars indicate 95% confidence intervals), in Example 6.

FIG. 9 shows cell viability of PER.C6® cell growth in scale-up of sparging conditions to 300 L (error bars indicate 95% confidence intervals), in Example 6.

### DETAILED DESCRIPTION OF THE INVENTION

The present invention relates to a process for large scale virus production. The methods disclosed herein are preferably adapted to suspension culture of mammalian host cells in a large scale bioreator where gas sparging becomes essential to provide adequate aeration through the duration of the culture. The large scale methods of the present invention preferably include the use of a virus seed stock for infecting a host cell culture whereby the virus seed stock has been generated free of cell lysis reagents, such as the detergent Triton X-100 or Polysorbate 80. It is disclosed herein that infecting the cell culture with such a virus seed stock while also including increased levels of at least one respective shear-protecting compound in conjunction with sparging results in a reproducible, commercially viable large scale virus production process. A preferred but by no means limiting shear-resistant compound is a surfactant such as Pluronic®F-68, a non-ionic detergent block copolymer which has been shown to be an effective shear resistant compound (i.e., helping to protect the integrity of the host cell, especially after viral infection) in serum free mammalian growth culture media. It will be within the scope of this invention to utilize other surfactants, whether they be other block copolymers from the Pluronic® series, or other surfactants which provide the same level of protection to the infected host cell as does Pluronic®-F68 (PF-68). For example, Pluronic® copolymers with a higher hydrophilic-lipophilic balance (see Murhammer and Goochee 1988, Bio/Technology 6:1411-1418) may be candidates for the methodology described herein. Other potential compounds include but are not limited to hydroxyethyl starch, derivatives of cellulose, serum, tryptosephosphate, polyvinyl alcohol (PVA), bovine serum albumin, polyethylene glycol (PEG), and dextran, with emphasis on components which retain the serum-free nature of the culture medium. It is within the scope of the present invention to mix and match one to several to an even higher number of shear-protecting compounds so as to mimic the large scale virus production levels as well as the consistent, reproducible large scale virus production levels disclosed herein. To this end, any shear-protecting compound or reagent that is available to the skilled artisan which provides for, alone or in any reasonable combination, a level of protection to the pre- and/or post-infected host mammalian cell, when compared to the exemplified Pluronic® F-68, is a shear-protecting compound or combined formulation thereof which is contemplated to be within the scope of the present invention. Regarding the timing of adding a shear-protective compound, it is shown herein a shear-resistant/protective compound may be added at any time during the cell culture process up to the time of infection with the respective virus seed. Therefore, while it is a decision of convenience to add a shear-protective compound(s) pre-infection while cells continue to grow in the culture medium (e.g., from the start of the large scale culture), it is certainly reasonable and well within the scope of the disclosed methodology to add all or the bulk of the shear protective agent at or reasonable near the time of seeding the culture with the respective virus seed stock. For example, it would be reasonable to grow the culture for a time in a medium which contains a lower concentration of PF68 (e.g., around 0.5 g/L) and then increasing the concentration at the time of infection into the 1-2 g/L or higher range. Alternatively of course, the concentration of the shear-protective compound may be brought up to post-infections levels at any stage leading up to the time at or around the time of infection of the cell culture with the respective virus stock seed.

The host cell for use in the method presented herein comprise any mammalian cell line which supports replication of the respective thermo-stable virus, especially any host cell line known in the art which will support infection and replication of a 1^{st} or 2^{nd} generation adenovirus vector. A preferred host cell is a host cell line which supports infection and replication of an E1 and/or and E1/E3 deleted recombinant adenovirus. As disclosed herein, such a replication-incompetent virus (such an Ad5gag, as exemplified herein) requires a helper cell line which complements the Ad5 E1 deletion. Any such cell line may be used to generate recombinant virus, with preferred, but not limiting, cell lines including 293 cells, PER.C6™ cells, 911 cells from a human embryonic retinal cell line (Fallaux et al. 1996, Human Gene Therapy 7: 215-222); E1-transformed amniocytes (Schiedner et al. 2000, Human Gene Therapy 11:2105-2116); an E1-transformed A549 cell line for a human lung carcinoma (Imler et al. 1996, Gene Therapy 3:75-84) and GH329: HeLa (Gao et al. 2000, Human Gene Therapy 11: 213-219). Such a cell line is transformed to support replication and packaging of a respective recombinant adenovirus, such as an E1 or E1/E3 deleted recombinant adenovirus. Additional cell lines which may be utilized in the present invention are again cell lines which have been adapted to act as host cells for a particular thermo-stable virus. It is preferable that the cell line be a continuous cell line and more preferable that the source of the cultured cells originate from a non-neoplastic tissue. It is also preferable that the source be mammalian, most likely from a primate origin, and especially of human origin. Again, a preferred cell line is a cell line which is useful for the propagation of an Ad E1 or E1/E3 deleted recombinant virus; a recombinant virus which compliment E1-deleted adenovirus vector included cell lines transfected with the gene encoding Ad E1 which have been selected for this transformed phenotype, such as 293 cells (epithelial cells from human kidney) and PER.C6™ (human embryonic retinoblasts). Other cell types include but are not limited to HeLa cells, A549 cells, KB cells, CKT1 cells, NIH/sT3 cells, Vero cells, Chinese Hamster Ovary (CHO) cells, or any eukaryotic cells which support the adenovirus life cycle.

As shown throughout this specification, several steps may be taken to resolve this problem for large scale applications for growing virus, such as adenovirus, under sparging conditions. A combination of steps includes (1) infecting the cell culture with virus seeds which are free of cell lysis components, and (2) adding at least one shear-protecting agent such as a surfactant (e.g., Pluronic®F-68) to the culture at a concentration of at least about 1 g/L or higher. The inclusion of these two steps allow for the production of robust virus lots that may be reproduced for large scale production runs, including bioreactor scale-up for adenovirus production in lots of 10,000 L or higher. The inclusion of a surfactant, such as Pluronic® F-68, in culture media at levels disclosed herein is not useful if a virus seed stock is used which contains even small amounts of cell lysing materials. Since virus production is reduced and inconsistent under sparging conditions at large scale when using different medium lots and virus seeds containing cell lysis materials, the additional step of generating and using virus seed stocks free of cell lysing agents become required. To this end, virus seeds were prepared without use of any cell-lysing components. By implementing these two measurements, virus productivity was improved to the same level as a non-sparged small scale control and variations were greatly minimized. Consistent virus production was achieved among different batches using different medium lots. Overall process robustness was significantly improved, demonstrating consistent virus productivity under the worst case scale down gas sparging conditions which would be used at 10,000 L scale for Adenovirus production.

Adenovirus production in serum-free PER.C6™ cell culture is technology that has not been practiced routinely at large-scale, where gas sparging is required for oxygenation. At small-scale, surface aeration instead of gas sparging is generally used to maintain adequate oxygen supply and hence the sensitivity to sparging can be circumvented. At large-scale, gas sparging is often necessary for oxygenation and hence resolving issues with sensitivity to gas sparging for virus infected cell cultures are necessary to allow use of this convenient oxygen method for large-scale bioreactor design and virus production. Virus seeds are often prepared in the presence of cell lysing components, such as Polysorbate 80 or Triton X-100, which are used for effective cell lysis and virus release. These cell lysis reagents containing virus seeds are then used directly in culture without purification. To the best of the inventors present knowledge, there have been no reports on effects of using such seeds for virus infection. It is shown herein that under gas sparging conditions, Adenovirus productivity has been found variable and generally much lower than under surface aeration when employing virus seeds prepared using buffers containing one or more cell-lysing components (e.g. Polysorbate 80 or Triton X-100) at a concentration for cell lysis. A dose-dependent effect of the detergent containing lysis buffer is shown herein in experiments performed in small-scale bioreactors under gas sparging conditions.

As noted above, gas sparging remains a widely used and often necessary method for oxygenation and CO₂ removal in large-scale mammalian cell culture. To protect cells from damage by gas sparging, the surfactant Pluronic® F-68 is generally used. However, virus infected cells may behave differently from uninfected cells towards gas sparging. Even though Pluronic® F-68 can protect uninfected cells from damage due to gas sparging even at relatively low concentrations, Pluronic® F-68 may or may not protect infected cells from damage for virus production due to gas sparging depending on how virus seeds are prepared. It has been determined and disclosed herein that Adenovirus productivity is variable and generally much lower under gas sparging conditions than under surface aeration when virus seeds prepared using buffers containing one or more cell lysis compounds such as Polysorbate 80 or Triton X-100 are employed. Experiments performed in small-scale bioreactors under gas sparging conditions indicate a dose-dependent effect of the detergent containing lysis buffer. Virus productivity is impaired under gas sparging conditions in presence of such detergents. Cell lysing components, even in a small amount, may damage the cell membrane, which makes cells more sensitive to shear from agitation and gas sparging. They may also interact with culture medium additives, notably the shear protection agent, such as Pluronic® F-68. This may either deplete Pluronic® F-68 and/or impair its ability to prevent cell attachment to bubbles, and hence it may be unable to protect infected cells from sparging. The serum-free medium used for PER.C6™ cells and Adenovirus cultivation (293 SFM II, Invitrogen) contains 0.3 g/L Pluronic® F-68. Even though it is sufficient to protect uninfected PER.C6™ cells from damage, it does not effectively protect PER.C6™ cells infected using the virus seeds prepared in the presence of cell-lysis reagents under sparging conditions.

Adenovirus seeds are generally prepared by recovery of extra- and/or intracellular viruses of infected cell culture. Intracellular viruses can be recovered through cell lysis, and virus concentration can be achieved either by ultrafiltration or separation of virus-containing cells prior to lysis. In general, buffers containing detergents such as Polysorbate 80 or Triton X-100 or freeze/thaw are used to achieve cell lysis for virus release. The cell lysis procedure using detergents allows significant cell lysis and virus release. It is simple, scalable and widely used, but the resulting virus seeds contain a detergent(s). Unfortunately, the present inventors have found that the use of such popular formulations may or may not have effects on their subsequent use for virus propagation depending on culture conditions. On the other hand, the cell lysis procedure using freeze/thaw, even though it is simple and mostly used at small-scale, can not be easily scaled up. Thus, it is not practical for preparation of large-scale virus seeds, which are used to infect cultures at large-scale (1,000 L or larger).

It is disclosed herein that it is critical to use the virus seeds prepared without use of detergents for cell lysis. Therefore, the present invention also relates to methods of producing virus seed stocks, especially adenovirus seed stocks, which are free of cell-lysis components, which comprises first inoculating and culturing host cells in a cell growth medium free of cell lysis components, infecting the host cells with an adenovirus of any serotype, either of a wild type or recombinant form. The resulting adenovirus-infected host cells are cultured for an appropriate time and then the adenovirus is harvested from both infected host cells (intracellular adenovirus) as well as the cell culture medium (extracellular adenovirus) without the aid of any added cell-lysis component, and concentrating the harvested adenovirus, resulting in a unclarified adenovirus seed stock free of any cell-lysis product. To this end, a scaleable process has been developed for preparation of the Adenovirus seeds from infected PER.C6™ cell suspension cultures under aseptic conditions without use of cell-lysing additives.

A particular embodiment of the present invention relates to a scaleable process which comprises using mechanical shear for cell lysis and virus release without the use of cell-lysis components, and using hollow fiber filtration technology for virus concentration to reduce volume for storage. Tangential flow filtration is widely used in the bioprocessing industry for cell harvesting, clarification and concentration of products including recombinant proteins and viruses. The tangential flow design allows for high shear rates and turbulence in the vicinity of the membrane to obtain high mass transfer rates. The system is composed of three distinct process streams: the feed solution, the permeate and the retentate. Depending on applications, filters with different pore sizes are used. For cell harvesting and clarification of cell debris, filters with a pore size of 0.1 μm or larger are used. Either the retentate or the permeate is the product. Ultrafiltration is generally referred to filtration using filters with a pore size of smaller than 0.1 μm. Products are generally retained, while volume is reduced through permeation. The two most widely used geometries for tangential flow filtration in the biopharmaceutical industry are plate & frame and hollow fiber modules. Hollow fiber units for ultrafiltration and microfiltration were developed by Amicon and Ramicon in the early 1970s (Cheryan, M. Ultrafiltration Handbook), even though there are now multiple vendors including Spectrum and A/G Technology. The hollow fiber modules consist of an array of self-supporting fibers with a dense skin layer that gives the membranes its permselectivity. Fiber diameters range from 0.5 mm-3 mm. One of the main advantages of the hollow fiber modules is the ability to obtain high shear rates at the membrane wall (thus high mass transfer rate) due to relatively small diameter of fibers and high fluid velocity. Another advantage of the hollow fiber modules is availability of filters from very small membrane areas (ca. 16 cm²) to very large membrane areas (ca. 28 m²) that allows linear and simple scale-up.

For virus seed production a major consideration is aseptic processing, in which case filter modules need to be either autoclavable or steam-in-place capable. Spectrum Laboratories (CA) and A/G Technologies are major suppliers of such units-both suppliers provide polysulfone ultrafiltration modules from 50 kDa to 750 kDa nominal molecular weight cutoff (NMWC), which can be sterilized.

It is noted that other filtration technologies may be used to practice this portion of the present invention, several of which are exemplified herein. As an example, but certainly not a limitation, plate and frame technology can also be used in lieu of hollow fiber membranes. The inventors have recently developed a process that allows simultaneous lysis and clarification of virus bulks from adenovirus infected PER.C6™ cell culture. Microfiltration modules offered by Sartorius (Sartocon, 0.45 μm Hydrostart™) are used in a process where virus is collected in the permeate. Infected cells are lysed to release intra-cellular virus using shear generated in the rotary lobe pump and the microfiltration module at the same time. In order to maximize virus permeation and to minimize membrane fouling, the flux is controlled during the process to ca. 20 liters per hour per m² of membrane area. The permeate containing the virus is then concentrated for volume reduction by ultrafiltration cross flow filtration. The membrane for this step is selected to provide high flux while retaining the virus, such as a 300 kDa NMWC polyethersulfone membrane (Sartorius).

In a preferred aspect of this portion of the present invention, Adenovirus seeds are prepared under aseptic conditions from infected PER.C6™ cell suspension cultures. The infected cell culture is harvested at ca. 48 hours post infection by initiating recirculation through a hollow fiber ultrafiltration system with a molecular weight cut off smaller than the virus to be retained. A small lumen diameter is selected to maximize the wall shear for effective cell lysis and virus release. During the recirculation, intracellular viruses are released into the supernatant due to cell lysis by mechanical shear, and concentrated along with extracellular viruses, which are present in the supernatant prior to cell lysis. At the end of the concentration, the permeate is closed and additional recirculation may be used to ensure complete cell lysis and virus release. As a result, concentrated and unclarified virus seeds are prepared. Such an approach not only simplifies the concentration process for volume reduction, as ultrafiltration is done with unclarified virus culture harvests, but also ensures complete cell lysis for virus release at the same time without use of cell-lysis reagents. The unclarified virus seeds prepared using this process at lab-scale and at 240 L scale have been successfully tested for subsequent infection with consistent virus productivity. As noted earlier, the present invention also relates to methods of producing virus seed stocks, especially adenovirus seed stocks, which are free of cell-lysis components. This portion of the invention comprises first inoculating and culturing host cells in a cell growth medium free of cell lysis components, infecting the host cells with an adenovirus of any serotype, either of a wild type, modified (e.g., such as an attenuated virus) or more likely, a recombinant form. A preferred embodiment in the large scale methodology of the present invention is the use of the shear-resistant compound is Pluronic®-68 (PF-68). It is shown herein that PF-68 may be used without problem at levels at least as high as 10 g/L. In view of these teachings, the skilled artisan may increase PF-68 levels as seen fit. Therefore, usable ranges for PF-68 are at least from about 0.3 to about 10 g/L, and even higher, as can be tested by the artisan. A more applicable range may be a PF-68 concentration from 1 g/L to 2 g/L. Also, gas sparging conditions (utilized in combination with PF-68 and virus seeds free of cell lysing components) are shown herein to coincide with sparging rates required for large scale production runs. Therefore, the methods of large scale virus production will utilize gas sparging at a rate corresponding to a rate of 0.001 to 0.05 VVM, with the most preferable rate of 0.01 VVM.

A preferred methodology for generating virus seed stocks free of any cell-lysis components, including but not limited to adenovirus seed stocks, is the use a hollow fiber ultrafiltration to simultaneously harvest virus from the infected cell culture, lyse the host cells mechanically, and concentrate the available virus particles, such as adenovirus particles, providing for a concentrated, unclarified virus seed stock useful in infecting large scale cell culture for virus production. As noted herein, the adenovirus may be further processed in any number of ways that are known in the art. For example, the cell lysis component-free adenovirus may be concentrated, resulting in an unclarified, concentrated adenovirus seed stock free of any cell-lysis reagents. Also, clarification and virus release may be a combined step, followed by concentration of the remaining virus volume to generate a clarified virus seed stock free of any cell-lysing components. Other potential avenues for generating the final virus seed stocks of the present invention include but are in no way limited to (1) a lysis/concentration stage which results in an unclarified virus seed (2) a lysis/concentration stage followed by clarification via any known methodology, such as filtration or centrifugation, which results in a clarified virus seed; (3) lysis/clarification followed by concentration of the clarified virus seed, and/or (4) lysis/clarification for unconcentrated virus seed. The above mentioned protocols are conducted under aseptic conditions. In the event of any non-aseptic processing a sterile filtration step may follow. Any method or combination of methods may be used to prepare the virus seeds generated free from cell lysis components, including but not limited to a clarified virus seed, an unclarified virus seed, or even an unclarified virus seed which is then subject to a later clarification step. Therefore, it is certainely within the purview of the present teachings for the skilled artisan to practice non-aspectic steps during the virus recovery process and then follow up with a sterile filtration step, or in the alternative, to generate a non-aseptic seed in itself.

The present invention also relates to virus seed stocks which are devoid of any cell-lysing components, such as detergents, including but not limited to adenovirus seed stocks.

The present invention also relates to virus seed stocks which are devoid of any cell-lysing components, such as detergents, including but not limited to adenovirus seed stocks, of which were generated by the methods disclosed herein.

As noted above, a preferred embodiment of the present invention relates to adenovirus seed stocks which are devoid of any cell-lysing components, such as detergents. An adenovirus seed stock may be a stock of any wild type and/or recombinant serotype of adenovirus known in the art.

While specific cell culture and virus production conditions are disclosed within the Example sections herein, it will be within the purview of the artisan of ordinary skill to use the teachings of the present invention to optimize for use in a respective host cell/virus culture system. It is also within the scope of the present invention to alter or manipulate culture conditions, media components and other such steps or methods which are known to the artisan which may be used in combination with virus stock seeds free of cell-lysis components as well as using elevated levels of a surfactant in the culture medium, especially after virus infection. Regardless of the specific parameters adapted, incorporation of these teachings allows for consistent, reproducible production of virus in a large scale commercial setting. While the present invention is exemplified for a recombinant adenovirus 5 serotype, it is within the scope of the present invention to apply these teachings to other adenovirus serotypes. It will also be within the purview of the skilled artisan to apply these teachings to other viruses, including but not necessarily limited to any intracellular viruses produced in mammalian cell culture, such as Rotavirus in Vero cells. In other words, this technology will be useful in a scenario where cell lysis is required by virus release.

A specific embodiment of the present invention relates to a process for the large scale production of adenovirus within a sparged culture system. As noted above, the methods disclosed herein as applied to any wild type or recombinant adenovirus serotype are preferably adapted to suspension culture of mammalian host cells in a large scale bioreator with gas sparging to provide adequate aeration. This large scale method also preferably includes the use of adenovirus seed stocks which have been generated free of cell lysis reagents and the inclusion of a shear-protecting compound(s) to offset deleterious effects by gas sparging conditions on infected host cells. Again, a preferred but by no means limiting shear-protecting compound is a non-ionic surfactant, such as the surfactant Pluronic®F-68. Therefore, the present invention relates to a robust process for large-scale production of recombinant Adenovirus vectors in serum-free cell culture under gas sparging conditions. As noted herein, several measures were taken to allow for reproducible virus production protocols at large scale volumes. First, a shear-protecting compound such as Pluronic®F-68 concentration is increased to 1 g/L or higher to improve process robustness. Second, virus seeds are prepared without use of any cell-lysing compounds, such as detergents or enzymes know in the art to lyse cells (e.g., Triton X-100, Polysorbate 80). As taught herein, by implementing these two measurements, virus productivity increases and large scale culture run to run variations are minimized. Consistent virus production has been achieved among different batches using different medium lots. Overall process robustness has been significantly improved. This process has been demonstrated in multiple batches in a 300 L bioreactor with consistent virus productivity using a gas sparging rate anticipated for much larger bioreactor scale for Adenovirus production (1,000 L or higher). To this end, a particular embodiment of the present invention is exemplified herein, namely the large-scale production of a recombinant Ad5gag virus in serum-free cell culture under gas sparging conditions with Pluronic®F-68 added to the culture at a concentration of at least about 1 g/L while adenovirus seeds used to infect the host cell culture are prepared without use of any cell-lysing reagents, such as cell lysing detergents or enzymes. Again, the inclusion of these two steps allow for the production of robust virus lots that may be reproduced for large scale production runs, including bioreactor scale-up for adenovirus production in lots of 1,000 L or higher.

Therefore, a scaleable process is developed for preparation of the Adenovirus seeds from infected PER.C6™ suspension cultures under aseptic conditions without use of detergents for cell lysis. Such a scaleable process is necessary so that virus seeds can be prepared at large-scale (1,000 L or larger). At small scale, freeze/thaw is generally used for cell lysis and virus release without use of detergents. Even though it is simple, it can not be scaled up for preparation of virus seeds at 1,000 L or larger. In the inventor's labs, a scaleable process is developed using mechanical shear for cell lysis and virus release without use of detergents, and using hollow fiber filtration technology for virus concentration to reduce volume for storage. In particular, a process using hollow fiber ultrafiltration technology was developed to provide significant cell lysis for virus release and to reduce volume for virus concentration at the same time. To prepare adenovirus seeds under aseptic conditions from infected PER.C6™ cell suspension cultures, infected cell culture is harvested at ca. 48 hours post infection by initiating recirculation through a hollow fiber ultrafiltration system with a molecular weight cut off smaller than the virus to be retained. A small lumen diameter is selected to maximize the wall shear for effective cell lysis and virus release. During the recirculation, intracellular viruses are released into the supernatant due to cell lysis by mechanical shear, and concentrated along with extracellular viruses, which are present in the supernatant prior to cell lysis. At the end of the concentration, the permeate is closed and additional recirculation may be used to ensure complete cell lysis and virus release. As a result, concentrated and unclarified virus seeds are prepared. Such an approach not only simplifies the concentration process for volume reduction, as ultrafiltration is done with unclarified virus culture harvests, but also ensures complete cell lysis for virus release at the same time without use of detergents. The unclarified virus seeds prepared using this process at lab-scale and at 240 L scale have been successfully tested for subsequent infection with consistent virus productivity.

The following non-limiting Examples are presented to better illustrate the invention.

### EXAMPLE 1

### Adenovirus Seed Stock Preparation—Freeze Thaw

Infected culture with a recombinant Adenovirus vector encoding HIV transgene gag is harvested at 48 to 72 hours post virus infection for virus seed stock preparation. The infected cell suspension is collected and spun down by centrifugation at 100 g to 1000 g for 10-20 minutes. The resulted cell pellet is then resuspended in a small volume of spent or fresh medium for a concentration factor of 10 to 20 from the original harvested cell suspension. The concentrated cell suspension is frozen and thawed three times to break up cells and facilitate virus release. The cell debris is then removed by centrifugation at 500 to 1000 g for 10-30 minutes and the resulted cell lysate supernatant is the virus seed stock. The virus seed stock is then dispensed into small aliquots and stored at −70° C. for future use.

### EXAMPLE 2

### Adenovirus Seed Stock Preparation: Hollow Fiber Ultrafiltration—Lab Scale

PER.C6™ cells cultured in 20 L Wave Bioreactors with a 10 L working volume were infected with a recombinant Adenovirus vector encoding a HIV transgene nef at a multiplicity of infection (MOI) of 290 viral particles per cell (vp/cell) and a viable cell concentration of 0.72×10⁶ cells/ml. Forty nine hours post infection (hpi), two wave bioreactors at a total cell concentration of 0.66×10⁶ cells/ml with 81% viability were harvested. At the time of the start of concentration, 20.4% of the virus was in the supernatant per anion exchange HPLC (AEX) assays.

A polysulfone hollow fiber membrane from Spectrum with a surface area of 0.39 m² and a pore size of 0.05 μm was used to lyse and concentrate these harvested cultures for preparation of a virus seed. The unit operation was performed at a constant crossflow rate of 4.4 L/minute and a transmembrane pressure (TMP) of ca. 7 psig under aseptic conditions with the hollow fiber filter sterilized by autoclaving and flushed with water for injection (WFI) before and after autoclaving to remove extractables.

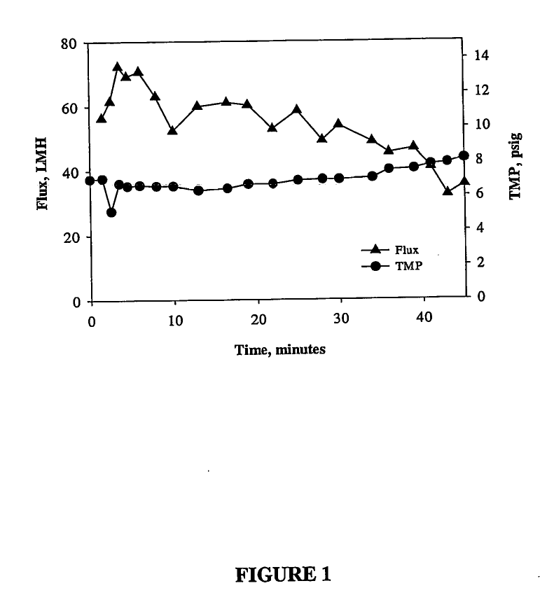

The entire harvest, ca. 17 L was concentrated 15 fold and the average flux was 55 L/m²/h (LMH), while cells were lysed for virus release. FIG. 1 shows the flux and TMP profile throughout the unit operation. Once the desired concentration factor was achieved, the retentate was further recirculated through the system (with the retentate valve remaining restricted and the permeate side of the membrane completely closed) for 20 minutes.

Table 1 summarizes the virus and material balance. The virus released in the supernatant at the end of the concentration was 86%. Based on the recovered volumes, 72% of the available virus was recovered in the retentate supernatant. Approximately 1 L of unclarified and concentrated virus seed at 2.84×10¹⁴ VP/L was obtained for further use.

TABLE 1
Material balance and anion exchange (AEX) assay results at lab-scale

| | AEX Virus | | |
|---|---|---|---|
| | Particle | | Total Virus |
| | Concentration | Volume | Particles |
| Sample | (×10¹⁰ VP/ml) | (ml) | (×10¹⁴ VPs) |
| Feed (total virus) | 2.52 | 17150 | 4.32 |
| Feed - Supernatant | 0.51 | 17150 | 0.88 |
| Permeate | <LOD | 15950 | N/A |
| Retentate (total | 33.2 | 1093 | 3.62 |
| virus | | | |
| Retentate - | 28.4 | 1093 | 3.11 |
| Supernatant | | | |

### EXAMPLE 3

### Adenovirus Seed Stock Preparation—Hollow Fiber Ultrafiltration—Large Scale

PER.C6™ cells cultured in a 300 L bioreactor with 240 L working volume were infected with a recombinant Adenovirus encoding a HIV transgene gag at a viable cell concentration of 0.59×10⁶ cells/ml at a multiplicity of infection (MOI) of 280 vp/cell. Fifty two hours post-infection (hpi), the bioreactor, at a total cell concentration of 0.55×10⁶ cells/ml with 55% viability was harvested. At the time of the start of this unit operation, 25% of the virus was in the supernatant (per AEX).

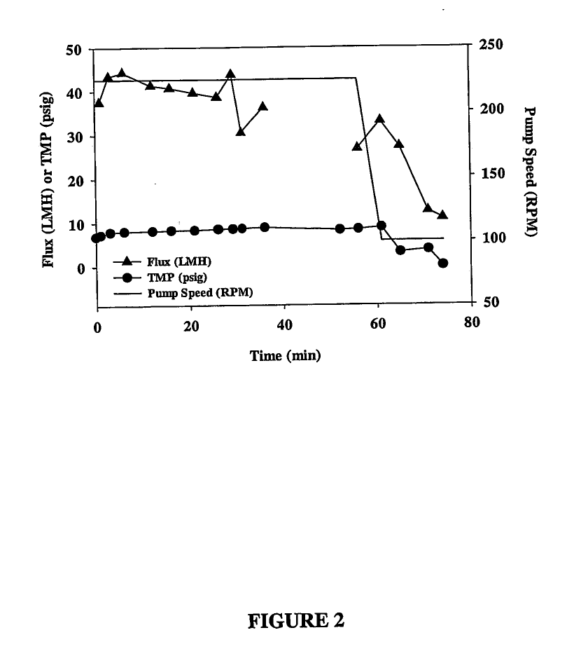

A polysulfone hollow fiber membrane from A/G Technology with a surface area 5.6 of m² and a pore size of 300 kDa was used to lyse and concentrate 200 L of harvested culture for preparation of a virus seed. The unit operation was performed at a crossflow rate of 27 LPM and TMP maintained at 7.5 psig under aseptic conditions with the hollow fiber sterilized using steam-in-place (SIP) as per manufacturer's recommendations and flushed with WFI before sterilization to remove extractables. The 200 L harvest was concentrated 32 fold and the average flux during the process was 33 LMH. Sixty minutes into the operation, the cross-flow rate was reduced to prevent foaming and air entrainment, which resulted in a reduction of both the TMP and flux. FIG. 2 shows the flux and TMP profile throughout the unit operation. Once the desired concentration factor was achieved, the retentate was further recirculated through the system (with the retentate valve open and the permeate side of the membrane completely closed) for 5 minutes.

Table 2 summarizes the virus and material balance. The virus released in the supernatant at the end of the concentration was 79.9%. Based on the recovered volumes, 58.7% of the available virus was recovered in the retentate supernatant. Approximately 4.4 L of unclarified and concentrated virus seed at 14.4×10¹⁴ VP/L was obtained for further use.

TABLE 2
Material balance and anion exchange (AEX) assay results at large scale

| | AEX Virus | | |
|---|---|---|---|
| | Particle | | Total Virus |
| | Concentration | Volume | Particles |
| Sample | (×10¹⁰ VP/ml) | (L) | (×10¹⁴ VPs) |
| Feed (total virus) | 5.60 | 193.3 | 108.2 |
| Feed - Supernatant | 1.40 | 193.3 | 27.1 |
| Permeate | N/R | 184.3 | — |
| Retentate (total | 180.60 | 4.4 | 79.5 |
| virus) | | | |
| Retentate - | 144.30 | 4.4 | 63.5 |
| Supernatant | | | |

### EXAMPLE 4

### Adenovirus Seed Stock Preparation: Microfiltration and Ultrafiltration Using Plate and Frame Membranes—Lab Scale

PER.C6™ cells cultured in a 300 L bioreactor with 240 L working volume were infected with a recombinant Adenovirus encoding a HIV transgene gag at a viable cell concentration of 0.51×10⁶ cells/ml at a multiplicity of infection (MOI) of 70 vp/cell. Forty eight hours post-infection (hpi), the bioreactor, at a total cell concentration of 0.64×10⁶ with 63% viability was harvested. At the time of start of this unit operation, 29.7% of the virus was in the supernatant (per AEX).

Two regenerated cellulose membrane slices from Sartorius with a combined surface area of 0.24 m² and a pore size of 0.45 μm were used to lyse and clarify 20 L of harvested culture. This unit operation consisted of two steps, lysis and clarification. The lysis step was performed at a crossflow rate of 4.0 LPM with the permeate port closed. Following lysis, the clarification was peformed at a crossflow rate of 4.0 LPM with a permeate flux controlled at 21.4 LMH. The membranes were sterilized using steam-in place (SIP) as per manufacturer recommendations and flushed with WFI before and after sterilization to remove extractables.

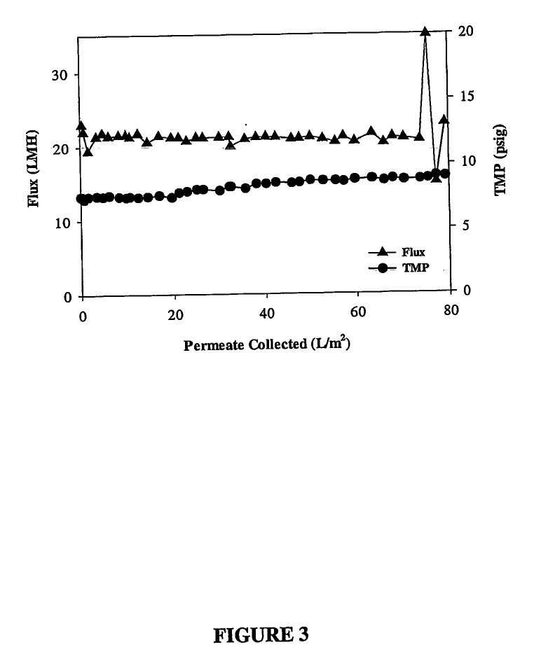

About 19 L was collected as permeate and the transmembrane pressure (TMP) increased from 7.5 psig to 9.0 psig during the process. FIG. 3 shows the flux and TMP profile throughout the clarification step. Table 3 summarizes the virus and material balance. The virus released in the supernatant at the end of the 40 minute lysis step was 92%. Based on the recovered volumes, 65.7% of the available virus was recovered in the permeate. A similar lysis/clarification experiment was done using the same harvest to obtain additional volume and the permeate from both experiments were pooled.

TABLE 3
Material balance and anion exchange (AEX) assay results for clarification
Using 0.45 μm regenerated cellulose Sartorius membranes at lab scale

| | Virus Particle | | Total Virus |
|---|---|---|---|
| | Concentration | | Particles |
| Sample | (×10¹⁰ VP/ml) | Volume (ml) | (×10¹⁴ VP) |
| Feed (total virus) | 3.43 | 20851 | 7.15 |
| Feed - | 3.16 | 20851 | 6.59 |
| Supernatant | | | |
| Retentate - | 9.84 | 1829 | 1.80 |
| Supernatant | | | |
| Permeate | 2.47 | 19010 | 4.70 |

A polyethersulfone (PES) membrane slice from Sartorius with a surface area of 0.14 m² and a pore size of 300 kDa was used to concentrate 32 L of pooled permeate for the preparation of virus seed. The unit operation was performed at 1.4 LPM and TMP was maintained at ca. 9.1 psig. The membrane was sterilized using steam-in place (SIP) as per manufacturer recommendations and flushed with WFI before and after sterilization to remove extractables.

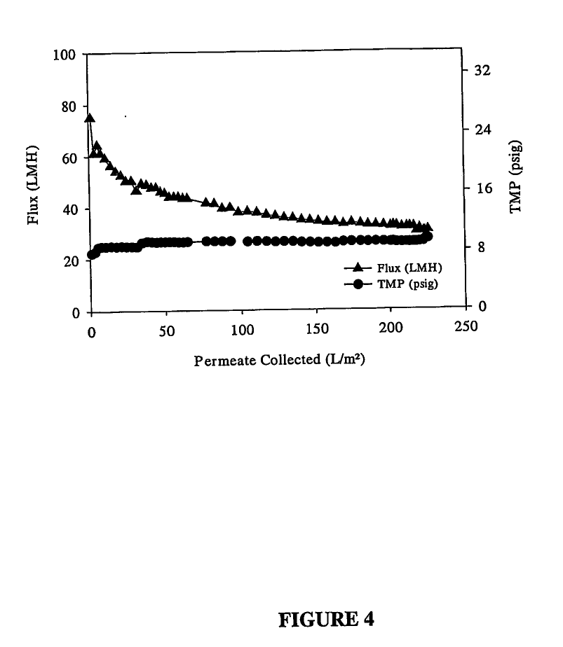

The 32 L permeate was concentrated 18.8 fold and the average flux during the process was 38.2 LMH. FIG. 4 shows the flux and TMP profile throughout the unit operation. Table 4 summarizes the virus and material balance. Based on the recovered volumes, 84% of the available virus was recovered in the retentate from the concentration step. About 1.7 L of clarified and concentrated virus seed at ca. 4.0×10¹⁴ VP/L was obtained for further use.

TABLE 4
Material balance and anion exchange (AEX) results for concentration
using 300 kDa PES Sartorius membrane at lab scale

| | Virus particle | | Total Virus |
|---|---|---|---|
| | concentration | | particles |
| Sample | (×10¹⁰ VP/ml) | Volume (ml) | (×10¹⁴ VP) |
| Feed | 2.6 | 32380 | 8.33 |
| Permeate | Below LOD | 31570 | — |
| Retentate | 40.4 | 1725 | 6.96 |

### EXAMPLE 5

### Sensitivity of PER.C6™ Cells to Sparging During Adenovirus Infection

This Example section shows the sensitivity of infected PER.C6™ cells to sparging and its effect on production of a recombinant adenovirus encoding HIV transgene gag (Ad5gag), as described in a series of experiments conducted in 2 L stirred tank bioreactors. Early studies indicated that virus productivity of sparged cultures was lower than surface aerated roller bottle and 2 L stirred tank bioreactor cultures. To mitigate the damaging effect of sparging, the concentration of Pluronic®68 (PF-68) in culture was increased. Cell growth rate and metabolism were found to be unaffected by PF-68 at concentrations up to 10 g/L. The presence of low concentrations of a virus seed buffer containing a cell lysis reagent was identified as a potential cause of sparging damage. The removal of the virus seed buffer (i.e., without cell-lysing components) and addition of PP-68 was then successfully demonstrated as a solution to the sparging problem with three different medium lots. This solution was confirmed in an infected culture at the 300 L scale under worst case sparging conditions. Therefore, based on these results, an increase of the PF-68 concentration to 1 g/L and using virus seeds formulated without use of any cell-lysing agents is useful for large scale adenovirus production processes, such as production scales of 1,000 L and 10,000 L, and upward.

Materials and Methods: Cell Line and Maintenance—PER.C6™ cells utilized in all Example sections have been adapted to suspension culture under serum-free conditions and are routinely maintained in 293 SFM II (Invitrogen, Grand Island, N.Y.) supplemented with 6 mM L-glutamine (Biowhittaker Inc., Walkersville, Md.) in a stirred bioreactor. 293 SFM II contains 0.3 g/L PF-68, additional PF-68 (Invitrogen, Grand Island, N.Y.) was supplemented when desired.

Virus Seed Stock—Ad5gag was amplified in PER.C6® cells. Virus seed was stored in a buffer containing 5 mM Tris, 1 mM MgCl₂, 75 mM NaCl, 5% (w/v) Sucrose, and 1% (w/v) Polysorbate-80 (Buffer A) or spent (or fresh) culture medium. The Buffer A virus seed stock was used for all experiments unless otherwise noted.

Bioreactor System—Small-scale bioreactor experiments were conducted in B. Braun Biostat MD twin bioreactor systems (B. Braun Biotech, Allentown, Pa.) at a working volume of 2 L.

Preparation of Virus Samples for Quantification—Total virus concentration was determined by either the sum of virus in the supernatant (Sup or S) and clarified cell lysate (CL) or the virus concentration in Triton X-100 lysed (TL) samples. Sup samples were prepared by centrifugation of harvested culture at 1800×g for 20 minutes and collecting the supernatant. The remaining cell pellet was resuspended and concentrated 10-fold in Buffer A containing 1% PS-80 and lysed via 3 times freeze and thaw, followed by centrifugation for removal of cell debris. TL whole broth samples were prepared by adding Triton X-100 to a final concentration of 0.1% (w/v) and stirring for one hour at 150 RPM. Samples were then clarified by collecting the supernatant after centrifugation at 1800×g for 20 minutes. Sup, CL, and TL samples were all stored at −70° C. prior to analysis.

Analytical Methods—Cell concentrations were determined with a hemocytometer and viability was obtained by trypan blue exclusion. Viral concentrations were detected by anion exchange HPLC (AEX assay), using a technique developed by Shabram et al. (1997, Human Gene Therapy 8:453-465).

Experiment #1: Effect of Sparging on Virus Production in Bioreactors and Roller Bottles—Pour 2 L bioreactors, 1 sparged and 3 unsparged, were infected with the virus seed described above at the same MOI. After infection 100 ml of culture was transferred from the sparged bioreactor to a roller bottle (RB) and gassed with 5% CO₂ in air for 30 seconds. At 48 hours post infection (hpi) cultures were sampled for cell concentration, viability, metabolites, and virus concentration (S and CL samples only).

Experiments #2 & 3: Toxicity of PF-68 on PER.C6™ Cell Growth—The purpose of these experiments was to identify the concentration limit of PF-68 that can be used for cell growth. Experiment #2 consisted of three unsparged bioreactors, inoculated in medium supplemented with PF-68 to final concentrations of 0.3 (none added), 1.0, and 2.0 g/L. Experiment #3 incorporated two sparged reactors, inoculated in medium supplemented with PF-68 to final concentrations of 1.0 and 10 g/L. All bioreactors were sampled daily for cell enumeration, viability, and metabolite concentrations.

Experiment #4: Effect of Concentration of buffer A containing 1% PS-80 on Virus Production—The purpose of this investigation was to determine the effects that increased amounts (or absence) of Buffer A would have on the virus production process. Seven bioreactors (six sparged) were inoculated at 2.0×10⁵ cells/ml and infected with the same MOI. The concentration of buffer A in the bioreactor was varied by spiking in buffer A or using virus seed formulated in spent or fresh culture medium. Amounts used were 0×, 1×, 7×, and 21× (1×=0.025% v/v). The non-sparged control had a final buffer A concentration of 1×. Two additional reactors had PF-68 concentrations of 1.0 g/L and buffer concentrations of 0× and 1×. All bioreactors were sampled for cell count, viability, and virus concentration (S, CL, and TL) at 48 hpi.

Experiment #5: Testing the Robustness of a Potential Solution to the Sparging Issue—This experiment shows that there is no difference in the virus productivity of sparged and non-sparged cultures when the PF-68 concentration is increased to 1 g/L and the Buffer A is removed from the virus seed. Six bioreactors were inoculated in medium with 1 g/L PF-68 and infected with virus seed in prepared in fresh medium (no buffer A added). Three different lots of 293 SFM II were used (one bioreactor sparged and one surface aerated for each lot). Bioreactors were sampled for cell count, viability, and virus concentration (S, CL, and TL) at 48 hpi.

Results and Discussion—The negative effects of sparging on virus production were observed in two early experiments (#1 and 2). In Experiment #1, virus productivity in the three non-sparged surface aerated bioreactors showed little variability (Table 5). Productivity in the sparged culture, however, was significantly lower than the control surface aerated roller bottle and the three unsparged 2 L bioreactors. In addition, the percentage of virus found in the supernatant was 10-fold higher in the sparged reactor, indicating a greater degree of cell lysis in the presence of sparging. Experiment #2 (Table 6) confirmed the harmful effect of sparging in a second comparison between a sparged bioreactor and control roller bottle. Again, considerably less virus was produced in the sparged bioreactor harvest at 48 hpi compared to the RB harvested at 72 hpi. Additional data demonstrates that virus concentration remains constant after 48 hpi.

TABLE 5
Effect of sparging on Adenovirus production

| | | S + CL | |
|---|---|---|---|
| | Sparge* | virus† | % in Sup |
| RB | N/A | 6.6 | 4% |
| 1 | No | 6.8 | 2% |
| 2 | No | 6.3 | 2% |
| 3 | No | 7.1 | 2% |
| 4 | Yes | 1.1 | 20% |

*Sparge rate: 0.05 VVM;
†Numbers indicate 10¹³ VP/L

TABLE 6
Confirmation of poor virus production with sparging

| | Harvest | CL | S | Total |
|---|---|---|---|---|
| | time | 10¹³ vp/L | | |
| RB | 72 hpi | 2.5 | 3.5 | 6.0 |
| Bioreactor* | 48 hpi | 1.0 | 1.7 | 2.7 |

*Sparge rate: 0.05 VVM

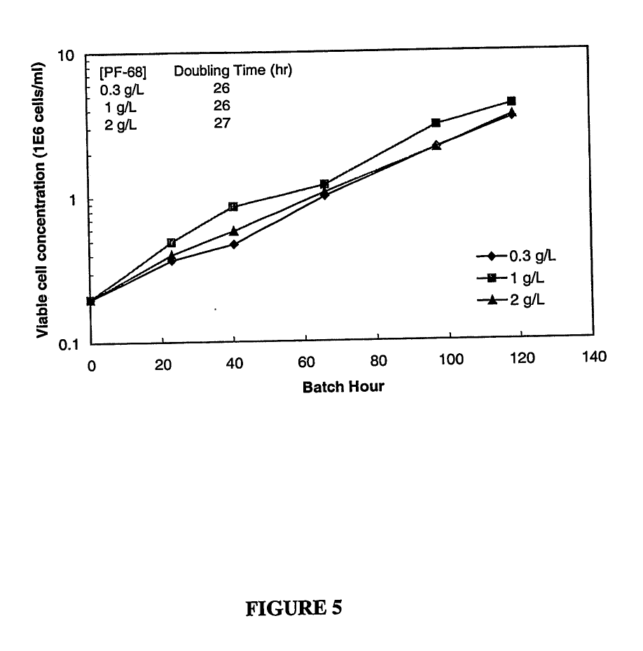

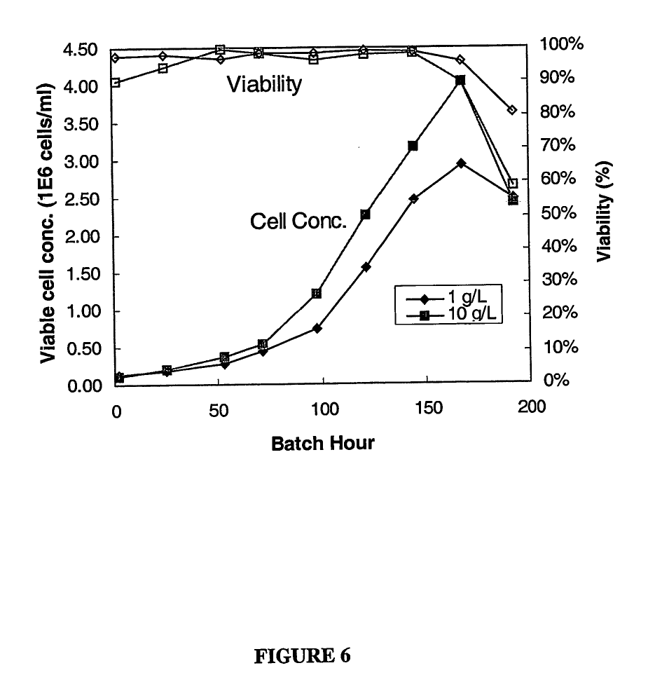

Effects of PF-68 concentration on cell growth and metabolism—Up to 2 g/L PF-68 is commonly used in sparged cell culture bioreactors. Before increasing the PF-68 concentration in the infection process it is necessary to observe any toxicity issues caused by high levels of PF-68 on PER.C6™ cells. Cell growth in various concentrations of PF-68 were tested under sparging (Experiment #4) and non-sparging (Experiment #3) conditions. Viable cell concentration is plotted on a semi-log scale in FIGS. 5 and 6. Doubling time is calculated from the slope and shown for comparison of cell growth rates. The figures indicate that there is no effect of PF-68 on cell growth rate at concentrations up to 10 g/L. As a result, a final PF-68 concentration of 1 g/L was used for the next experiment investigating virus production with sparging.

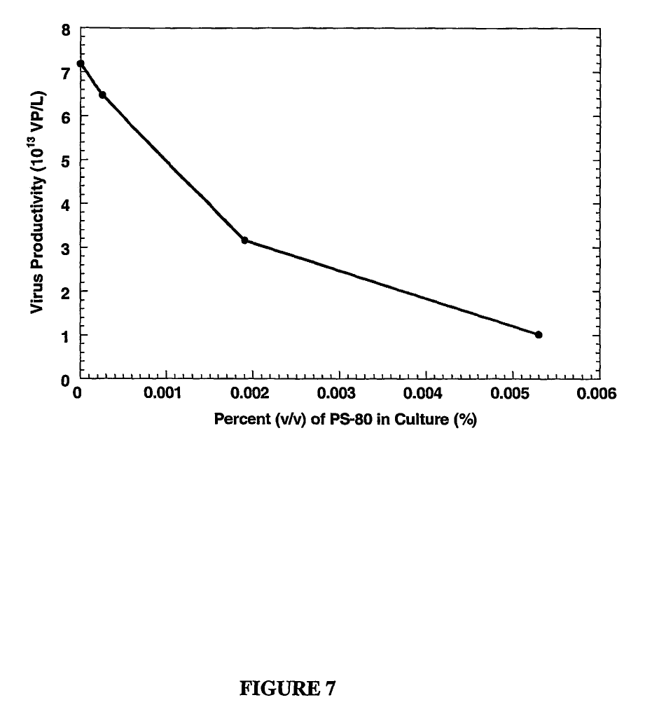

Experiment 4—Effect of buffer A containing 1% PS-80 on virus production in sparged cultures—For all previous experiments in this Example conducted at the 2 L and roller bottle scale, the virus seed used was formulated in Buffer A (which includes 1% PS-80). Upon addition to the reactor, this amounts to a final concentration of the buffer A in the culture of 0.025% v/v. Experiment #4 was designed to determine how this buffer affected virus production under sparging conditions. Table 7 summarizes virus production at varying concentrations of virus buffer (with and without 1 g/L PF-68) and in the non-sparged control. FIG. 7 demonstrates that virus production in sparged bioreactors is significantly affected by increased concentrations of virus buffer. It is clear that the combined effect of PF-68 addition and the removal of virus buffer from the seed significantly increase the robustness of the adenovirus production process.

TABLE 7
Virus concentration and data in Experiment #4

| | | Buff A | PF-68 | S + CL | TL | |
|---|---|---|---|---|---|---|
| | Sparge* | conc. † | conc. | virus ‡ | virus ‡ | % in Sup |
| 1 | No | 1X | 0.3 g/L | 7.7 | 9.4 | 22% |
| 2 | Yes | 0X | 0.3 g/L | 6.8 | 7.2 | 55% |
| 3 | Yes | 1X | 0.3 g/L | 5.5 | 6.5 | 62% |
| 4 | Yes | 7X | 0.3 g/L | 3.0 | 3.2 | 65% |
| 5 | Yes | 21X | 0.3 g/L | 0.7 | 1.0 | 48% |
| 6 | Yes | 0X | 1 g/L | 8.8 | 11 | 19% |
| 7 | Yes | 1X | 1 g/L | 4.3 | 4.9 | 50% |

† Final conc. of virus buffer in culture: 1X = ˜0.025% (v/v)
*Sparge rate: 0.05 VVM;
‡ Numbers indicate 10¹³ VP/L

Experiment 5—Testing a solution to the sparging issue—As a result of the data generated in Experiment #4, a higher PF-68 concentration of 1 g/L and infection with virus seed formulated in a medium containing no cell-lysis material was tested. In order to determine the robustness of this solution, sparged and non-sparged infections were tested head-to-head in three medium lots. For all three lots of 293 SFM II, the sparged bioreactors produced about the same amount of virus as the non-sparged controls. These results, summarized in Table 8 indicate that an increase to 1 g/L PF-68 and the removal of Buffer A containing 1% PS-80 from the virus seed solves the problem of sparging at the 2 L scale.

TABLE 8
Summary of results for Experiment #5

| | Medium | | S + CL | TL | % in |
|---|---|---|---|---|---|
| | lot | Sparge | virus* | virus* | Sup |
| 1 | A | Yes | 5.0 | 6.6 | 24% |
| 2 | A | No | 5.8 | 6.5 | 25% |
| 3 | B | Yes | 5.4 | 7.4 | 26% |
| 4 | B | No | 5.5 | 6.7 | 15% |
| 5 | C | Yes | 6.8 | 7.5 | 26% |
| 6 | C | No | 4.6 | 5.6 | 19% |

*Numbers indicate 10¹³ VP/L

### EXAMPLE 6

### Adenovirus Cultures Infected with Virus Seed Free of Cell-Lysis Reagent Under Sparging Conditions at Large-Scale

Recent experiments demonstrate that removal of 1% Polysorbate 80 from the virus seed and the addition of 0.7 g/L Pluronic F-68 overcome the deleterious effects of sparging at the 2 L scale. However, sparging at the large scale (300 L and up) will require much greater gas flow rates (see Table 9) than sparging at the small scale (2 L). This Example shows the sensitivity of PER.C6® cells to sparging at the 300 L scale under the methodology of the present invention.

TABLE 9
Scale-up of sparging in stirred vessel bioreactors

| | | | Prophetic @ |
|---|---|---|---|
| | 2 L | 300 L | 10,000 L |
| Working volume | 2 L | 250 L | 10,000 L |
| Liquid height | 0.15 m | 0.7 m | 3.7 m |
| Tank diameter | 0.13 m | 0.7 m | 1.9 m |
| Required sparge rate | 0.05 | 0.05 | 0.01 |
| (VVM) | | | |
| Q_{g}* (cm/min) | 0.7 | 3.5 | 3.7 |

*Superficial gas flow rate = Volumetric flow rate/surface area

Materials and Methods—Adenovirus seed for this study was prepared from a 10 L harvested virus bulk lysed and concentrated via hollow fiber ultrafiltration with no addition of lysis buffer in the same way as disclosed in Example 2. Aliquots were taken from this unclarified seed.

PER.C6® cultures from two 10 L bioreactors were pooled and used to inoculate a 300 L. Six 2 L volumes of culture were transferred from the 300 L vessel to six 2 L vessels approximately 2 hours after inoculation. Cells in both 2 L reactors and the 300 L bioreactor were infected with MRKAd5gag seed (the MRKAd5gag vector is described in WO 02/22080 hereby incorporated by reference in its entirety) at a MOI of ˜280 VP/cell. Approximately one hour after infection three 2 L volumes of infected culture were transferred from the 300 L vessel to three 2 L vessels. All bioreactors were sampled at 48 hours post infection (hpi). All 2 L vessels were sampled at approximately 72 hpi and 96 hpi as well. Viral concentrations from supernatant and Triton-X100 lysed samples (TL) were determined from the AEX assay. Experimental conditions such as sparge rate, time of inoculation, and seed used are summarized in Table 10.

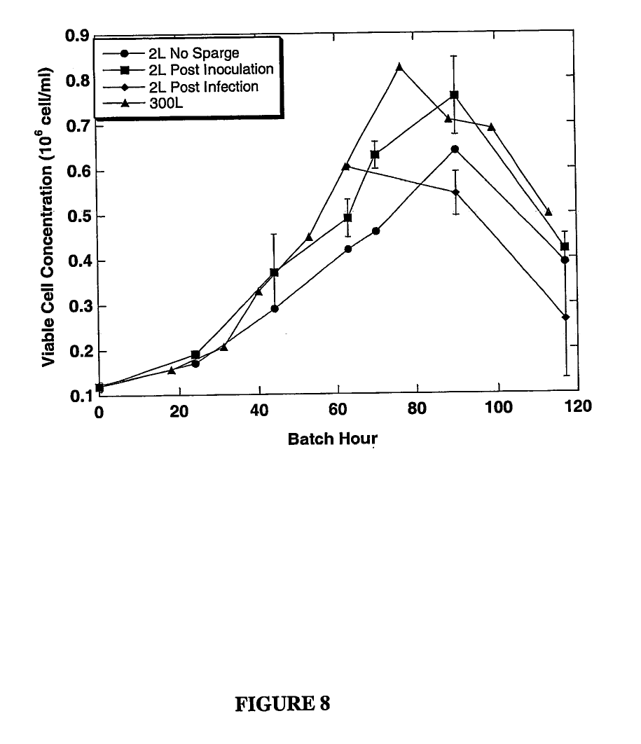

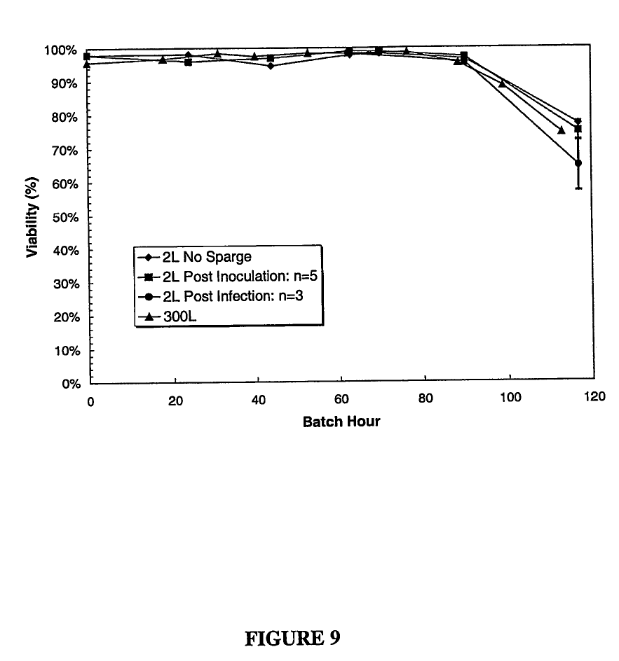

Results—The data presented in this Example section shows the ability of using a virus seed stock devoid of any cell-lysing materials in large scale production of adenovirus. FIG. 8 (cell growth) and FIG. 9 (cell viability) show that large scale (300 L) preparations provide similar results when using the virus seed stocks prepared as disclosed in the example 2 and 3. Virus production in the 300 L and 2 L control bioreactors are summarized in Table 10.

TABLE 10
Virus production 300 L bioreactor and 2 L controls under sparging
conditions.

| | Time cells | Sparge | | |
|---|---|---|---|---|
| | removed from | rate | AEX TL @ 48 | % in |
| Vessel ID | 300 L | (VVM) | hpi (1 × 10¹³ VP/L) † | Sup |
| 1 (300 L) | N/A | 0.05 | 9.1 | 11% |
| 2 (2 L) | Post-inoculation | No sparge | 6.6 | 7% |
| 3 (2 L) | Post-inoculation | 0.05 | 8.1 | 15% |
| 4 (2 L) | Post-inoculation | 0.05 | 8.6 | 14% |
| 5 (2 L) | Post-inoculation | 0.05 | 8.3 | 14% |
| 6 (2 L) | Post-infection | 0.05 | 6.8 | 28% |
| 7 (2 L) | Post-infection | 0.05 | 6.0 | 32% |
| 8 (2 L) | Post-infection | 0.05 | 7.5 | 14% |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

1. A method of large scale virus production, which comprises:
a) inoculating a cell growth medium with a population of host cells, wherein the cell growth medium contains an effective amount of a shear-protective compound;
b) culturing the host cells in the cell growth medium;
c) infecting the host cells in the cell growth medium with an aliquot of a virus seed stock, wherein the virus seed is essentially free of any cell-lysing component;
d) culturing the virus infected cells of step c) under gas sparging;
e) harvesting intracellular and/or extracellular virus from the host cells and cell growth medium; and,
f) purifying the harvested virus of step e).

2. A method of claim 1 wherein the shear-protective compound is selected from the group consisting of Pluronic®F-68, other Pluronic® copolymers, hydroxyethyl starch, derivatives of cellulose, serum, tryptosephosphate, polyvinyl alcohol (PVA), bovine serum albumin, polyethylene glycol (PEG), and dextran.

3. The method of claim 2 wherein the shear-protective compound is Pluronic®F-68.

4. The method of claim 3 wherein Pluronic®F-68 is present at a concentration from about 0.3 g/L and to about 10 μL.

5. The method of claim 4 wherein Pluronic®F-68 is present at a concentration from about 1 g/L to about 2 g/L.

6. The method of claim 1 wherein gas sparging is provided at a rate corresponding to a rate up to about 0.1 VVM.

7. The method of claim 6 wherein gas sparging is provided at a rate corresponding to a rate up to about 0.001 to 0.05 VVM.

8. The method of claim 2 wherein gas sparging is provided at a rate corresponding to a rate up to about 0.1 VVM.

9. The method of claim 8 wherein gas sparging is provided at a rate corresponding to a rate up to about 0.001 to 0.05 VVM.

10. The method of claim 3 wherein gas sparging is provided at a rate corresponding to a rate up to about 0.1 VVM.

11. The method of claim 10 wherein gas sparging is provided at a rate corresponding to a rate up to about 0.001 to 0.05 VVM.

12. The method of claim 4 wherein gas sparging is provided at a rate corresponding to a rate up to about 0.1 VVM.

13. The method of claim 12 wherein gas sparging is provided at a rate corresponding to a rate up to about 0.001 to 0.05 VVM.

14. The method of claim 5 wherein gas sparging is provided at a rate corresponding to a rate up to about 0.1 VVM.

15. The method of claim 14 wherein gas sparging is provided at a rate corresponding to a rate up to about 0.001 to 0.05 VVM.

16. A method of large scale adenovirus production, which comprises:
a) inoculating a cell growth medium with a population of host cells, wherein the cell growth medium contains an effective amount of a shear-protective compound;
b) culturing the host cells in the cell growth medium;
c) infecting the host cells in the cell growth medium with an aliquot of a adenovirus seed stock, wherein the adenovirus seed is essentially free of any cell-lysing component;
d) culturing the adenovirus infected cells of step c) under gas sparging;
e) harvesting intracellular and/or extracellular adenovirus from the host cells and cell growth medium; and,
f) purifying the harvested adenovirus of step e).

17. A method of claim 16 wherein the shear-protective compound is selected from the group consisting of Pluronic®F-68, other Pluronic® copolymers, hydroxyethyl starch, derivatives of cellulose, serum, tryptosephosphate, polyvinyl alcohol (PVA), bovine serum albumin, polyethylene glycol (PEG), and dextran.

18. The method of claim 17 wherein the shear-protective compound is Pluronic®F-68.

19. The method of claim 18 wherein Pluronic®F-68 is present at a concentration from about 0.3 g/L and to about 10 g/L.

20. The method of claim 19 wherein Pluronic®F-68 is present at a concentration from about 1 g/L to about 2 g/L.

21. The method of claim 16 wherein gas sparging is provided at a rate corresponding to a rate up to about 0.1 VVM.

22. The method of claim 21 wherein gas sparging is provided at a rate corresponding to a rate up to about 0.001 to 0.05 VVM.

23. The method of claim 17 wherein gas sparging is provided at a rate corresponding to a rate up to about 0.1 VVM.

24. The method of claim 23 wherein gas sparging is provided at a rate corresponding to a rate up to about 0.001 to 0.05 VVM.

25. The method of claim 18 wherein gas sparging is provided at a rate corresponding to a rate up to about 0.1 VVM.

26. The method of claim 25 wherein gas sparging is provided at a rate corresponding to a rate up to about 0.001 to 0.05 VVM.

27. The method of claim 19 wherein gas sparging is provided at a rate corresponding to a rate up to about 0.1 VVM.

28. The method of claim 27 wherein gas sparging is provided at a rate corresponding to a rate up to about 0.001 to 0.05 VVM.

29. The method of claim 20 wherein gas sparging is provided at a rate corresponding to a rate up to about 0.1 VVM.

30. The method of claim 29 wherein gas sparging is provided at a rate corresponding to a rate up to about 0.001 to 0.05 VVM.

31. A method of producing a virus seed stock free of cell-lysis components, comprising:
a) inoculating and culturing host cells in a cell growth medium free of cell lysis components;
c) infecting the host cells of step a) with a virus, resulting in virus-infected host cells;
d) culturing the virus-infected host cells;
e) harvesting intracellular and/or extracellular virus from the host cells and cell growth medium without the aid of any added cell-lysis component; and,
f) concentrating the harvested virus of step e), resulting in a unclarified virus seed stock.

32. The method of claim 31 wherein the viruses of step e) are released from host cells by non-mechanical shearing.

33. The method of claim 31 wherein the viruses of step e) are released from host cells by mechanical shearing.

34. The method of claim 33 wherein the mechanical shearing options are selected from the group consisting of hollow fiber ultrafiltration, plate and frame microfilitration, ultrasonics, high pressure homogenization, pumps, impinging jets and mechanical grinding.

35. The method of claim 31 wherein steps e) and f) are simultaneously accomplished by hollow fiber utltrafiltration of the infected cell culture for cell lysis and volume reduction.

36. An unclarified virus seed stock prepared by the method of claims 31-35.

37. A method of producing a adenovirus seed stock free of cell-lysis components, comprising:
a) inoculating and culturing host cells in a cell growth medium free of cell lysis components;
c) infecting the host cells of step a) with a adenovirus, resulting in adenovirus-infected host cells;
d) culturing the adenovirus-infected host cells;
e) harvesting intracellular and/or extracellular adenovirus from the host cells and cell growth medium without the aid of any added cell-lysis component; and,
f) concentrating the harvested adenovirus of step e), resulting in a unclarified virus seed stock

38. The method of claim 37 wherein the viruses of step e) are released from host cells by non-mechanical shearing.

39. The method of claim 37 wherein the viruses of step e) are released from host cells by mechanical shearing.

40. The method of claim 39 wherein the mechanical shearing options are selected from the group consisting of hollow fiber ultrafiltration, plate and frame microfilitration, ultrasonics, high pressure homogenization, pumps, impinging jets and mechanical grinding.

41. The method of claim 37 wherein steps e) and f) are simultaneously accomplished by hollow fiber utltrafiltration of the infected cell culture for cell lysis and volume reduction.

42. An unclarified virus seed stock prepared by the method of claims 37-41.

43. The method of claim 31 wherein the unclarified virus seed stock is further subjected to a clarification step.

44. A clarified virus seed stock prepared by the method of claim 43.

45. The method of claim 37 wherein the unclarified virus seed stock is further subjected to a clarification step.

46. A clarified virus seed stock prepared by the method of claim 45.
